(19) United States
(12) Patent Application Publication
(10) Pub. No.: US 2020/0261195 A1
(43) Pub. Date: Aug. 20, 2020

(54) KITS AND INSTRUMENTS FOR DELIVERING ANXIOLYTICS OR ANALGESICS TO THE MOUTH

(71) Applicants: Robert J. Harris, Bloomington, MN (US); Diane K. Gherity, Burnsville, MN (US)

(72) Inventors: Robert J. Harris, Bloomington, MN (US); Diane K. Gherity, Burnsville, MN (US)

(21) Appl. No.: 16/791,299

(22) Filed: Feb. 14, 2020

Related U.S. Application Data
(60) Provisional application No. 62/806,312, filed on Feb. 15, 2019.

(51) Int. Cl. A61C 15/04; A61C 15/02

(52) U.S. Cl. CPC A61C 15/046; A61K 31/352; A61C 15/02

(57) ABSTRACT

A dental instrument that is configured to deliver an anxiolytic or an analgesic to the oral cavity of a patient.

### CROSS-REFERENCE TO RELATED APPLICATION

This application claims priority to U.S. Provisional Application No. 62/806,312 that was filed on Feb. 15, 2019. The entire content of the applications referenced above is hereby incorporated by reference herein.

### BACKGROUND

Visiting a professional for a dental prophylaxis or oral procedure can cause anxiety for many patients. Currently there is a need for kits and instruments that can be used to administer anxiolytics and/or analgesics to the oral cavity of a patient prior to a dental procedure. Such kits and instruments would allow a patient to reduce anxiety prior to visiting the dentist or to reduce the level of pain experienced during a dental prophylaxis or oral procedure.

### SUMMARY

In one embodiment, the invention provides a dental instrument that is configured to deliver an anxiolytic or an analgesic to the oral cavity of a patient.

In another embodiment, the invention provides a kit comprising:

a) a dental instrument that is configured to deliver an anxiolytic or an analgesic to the oral cavity of a patient;

b) packaging material that contains an anxiolytic or an analgesic formulated for disposition (e.g. application) on the dental instrument;

c) instructions for disposing the anxiolytics or analgesic onto the dental instrument to provide a dental instrument that is configured to deliver the anxiolytics or the analgesic to the oral cavity of a patient; and

d) instructions for using the dental instrument that is configured to deliver the anxiolytics or the analgesic to the oral cavity of a patient prior to the patient visiting the dentist for a cleaning or an oral procedure, so that the anxiolytics or the analgesic is delivered to the patient and provides an anxiolytic effect and/or an analgesic effect during the cleaning or oral procedure.

In another embodiment, the invention provides a method for a patient to reduce anxiety prior to and/or while visiting a dentist, comprising the patient using a dental instrument that is configured to deliver an anxiolytics to the oral cavity of the patient prior to a dental prophylaxis or oral procedure.

### BRIEF DESCRIPTION OF THE FIGURES

FIG. 1 shows dental floss.

FIG. 2 shows a floss pick.

FIG. 3 shows a soft pick.

FIG. 4 shows a kit 100 that comprises a soft pick 101, packaging material (e.g. a vial) 102 that contains a liquid 103 that comprises CBD oil.

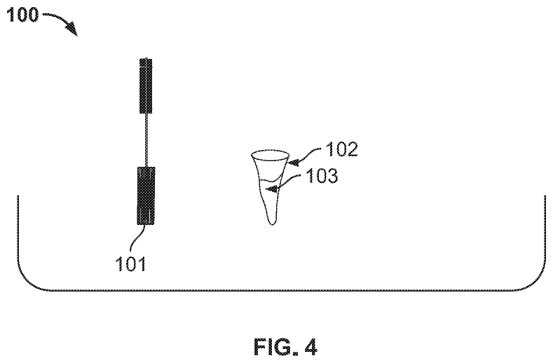

### DETAILED DESCRIPTION

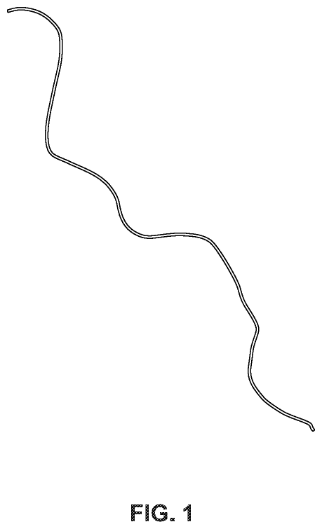

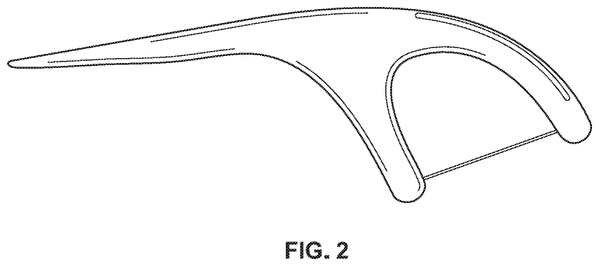

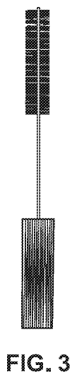

In one embodiment the dental instrument of claim 1, comprises dental floss (e.g. see FIG. 1). In one embodiment the floss is contained in a single use disposable applicator (e.g. a floss pick, see FIG. 2). In one embodiment, the dental instrument is dental floss. In one embodiment the dental instrument is a soft pick, see FIG. 3. One suitable soft pick is marketed by Sunstar Americas, Inc, under the name GUM Soft-Picks (e.g. Flex, Go-betweens, or Comfort Flex). As used herein, the term dental instrument does not include a syringe.

In one embodiment, the anxiolytic or the analgesic comprises a cannabinoid. In one embodiment, the anxiolytic or analgesic comprises tetrahydrocannabinol or an analog or derivative thereof. In one embodiment, the anxiolytic or the analgesic comprises cannabidiol (CBD) or an analog or derivative thereof. The term analog or derivative of tetrahydrocannabinol includes compounds that have similar chemical structures to tetrahydrocanibiniol (e.g. compounds that comprise the fused tricyclic ring structure of tetrahydrocanibiniol) and that possess anxiolytic and/or analgesic properties, including pharmaceutically acceptable salts and prodrugs thereof. The term analog or derivative of cannabidiol includes compounds that have similar chemical structures to cannabidiol (e.g. compounds that comprise the 2-cyclohexenyl-1,3,benzenediol ring structure of cannabidiol) and that possess anxiolytic and/or analgesic properties, including pharmaceutically acceptable salts and prodrugs thereof.

The action of disposing the anxiolytic or the analgesic onto the dental instrument to provide a dental instrument that is configured to deliver the anxiolytic or the analgesic to the oral cavity of a patient, can be carried out using any suitable method. For example, it can be carried out by dipping the dental instrument into a liquid or solid that comprises the anxiolytic or the analgesic, it can be carried out by rubbing the dental instrument with a liquid or solid that comprises the anxiolytic or the analgesic, or it can be carried out by inserting the dental instrument into a container (e.g. a vial) that contains a liquid or solid that comprises the anxiolytic or the analgesic, so that the dental instrument comes in contact with the liquid or the solid under conditions such that the anxiolytic or the analgesic are associated with the dental instrument to provide a dental instrument that is configured to deliver an effective amount of the anxiolytic or the analgesic to the oral cavity of a patient.

As used herein, the term patient includes any animal that might be exposed to a dental prophylaxis or an oral procedure. For example, in one embodiment, the term patient includes mammals (e.g., humans or a pets, such as dogs and cats). In another embodiment the term patient includes humans. In another embodiment the patient is a human.

What is claimed is:

1. A dental instrument that is configured to deliver an anxiolytic or an analgesic to the oral cavity of a patient.

2. The dental instrument of claim 1, which comprises dental floss.

3. The dental instrument of claim 2, wherein the floss is contained in a single use disposable applicator.

4. The dental instrument of claim 3, wherein the single use disposable applicator is a floss pick.

5. The dental instrument of claim 1, which is dental floss.

6. The dental instrument of claim 1, which is a soft pick.

7. The dental instrument of claim 1, wherein the anxiolytic comprises a cannabinoid.

8. The dental instrument of claim 1, wherein the anxiolytic comprises tetrahydrocannabinol, an analog or derivative of tetrahydrocannabinol, cannabidiol (CBD), or an analog or derivative of cannabidiol (CBD).

9. The dental instrument of claim 1, further comprising an analgesic.

10. A method for a patient to reduce anxiety prior to or while visiting a dentist, comprising the patient using the dental instrument of claim 1 prior to visiting the dentist.

11. A method for a patient to reduce pain while visiting a dentist, comprising the patient using the dental instrument of claim 1 prior to visiting the dentist.

12. A kit comprising:
a) a dental instrument;
b) packaging material that contains an anxiolytic or an analgesic formulated for disposition on the dental instrument;
c) instructions for disposing the anxiolytic or the analgesic onto the dental instrument to provide a dental instrument that is configured to deliver the anxiolytic or the analgesic to the oral cavity of a patient; and
d) instructions for using the dental instrument that is configured to deliver the anxiolytic or the analgesic to the oral cavity of a patient prior to the patient visiting the dentist, so that the anxiolytic or the analgesic is delivered to the patient and provides an anxiolytic effect and/or an analgesic effect prior to or during the patient visiting the dentist.

13. The kit of claim 12, which comprises dental floss or a single use disposable applicator.

14. The kit of claim 13, wherein the single use disposable applicator is a floss pick.

15. The kit of claim 14, wherein the dental instrument is a soft pick.

16. The kit of claim 12, wherein the anxiolytic comprises a cannabinoid.

17. The kit of claim 12, wherein the anxiolytic comprises tetrahydrocannabinol, an analog or derivative of tetrahydrocannabinol, cannabidiol (CBD), or an analog or derivative of cannabidiol (CBD).

18. A kit comprising:
a) a dental instrument selected from the group consisting of dental floss and a soft pick;
b) packaging material that contains tetrahydrocannabinol or cannabidiol formulated for disposition on the dental instrument;
c) instructions for disposing the tetrahydrocannabinol or the cannabidiol onto the dental instrument to provide a dental instrument that is configured to deliver the tetrahydrocannabinol or the cannabidiol to the oral cavity of a patient; and
d) instructions for using the dental instrument that is configured to deliver the tetrahydrocannabinol or the cannabidiol to the oral cavity of a patient prior to the patient visiting the dentist, so that the tetrahydrocannabinol or the cannabidiol is delivered to the patient and provides an anxiolytic effect and/or an analgesic effect prior to or during the patient visiting the dentist.

19. The kit of claim 18 wherein the dental instrument is dental floss.

20. The kit of claim 18 wherein the dental instrument is a soft pick.
